(19) United States
(12) Patent Application Publication
(10) Pub. No.: US 2009/0076452 A1
(43) Pub. Date: Mar. 19, 2009

(54) Disposable device for washing wounds and surgical sites

(75) Inventor: Pierino Moretti, Bastia di Rovolon (IT)

Correspondence Address: Themis Law, 7660 Fay Ave Ste H535, La Jolla, CA, 92037, US

(73) Assignee: EMMEBI S.R.L., Bastia di Rovolon (IT)

(21) Appl. No.: 11/719,751

(22) PCT Filed: Nov. 19, 2004
(86) PCT No.: PCT/IT2004/000642

§ 371 Date: Aug. 20, 2008

(51) Int. Cl. A61M 5/00

(52) U.S. Cl. 604/141

(57) ABSTRACT

The present invention concerns a new device for washing wounds and surgical sites that is equipped with a grip (1) with a generically linear shape, on which different interchangeable tips or terminals (7), having a straight or inclined direction, may be applied. The grip (1) includes an independent power source, such as a set of batteries. The pump (2) is actuated by a switch, which has a limited number of positions, for example, a position in which no electric power is supplied to the pump (2), a position in which electric power is supplied to the pump (2) and a position in which reduced power is supplied to the pump (2). The irrigating end of the internal pipe (7.5) is provided with a rotating sprinkling mechanism (7.5a) for spraying a washing liquid.

### FIELD OF THE INVENTION

The present patent concerns medical devices and in particular it concerns devices for washing wounds and surgical sites.

### BACKGROUND OF THE INVENTION

During surgery it is necessary to clean and wash accurately the operation site.

In particular, in surgery involving the bones and cartilage, such as removing and/or scraping bony material and reducing bony or cartilaginous portions, it is necessary to frequently clean the area of intervention and to suck up the washing liquid and the removed particles.

Devices are known for washing surgical sites and wounds, which comprise a grip in the shape of a gun, to the front of which a terminal or tip in the form of an irrigation and suction rod is connected.

Inside the grip there is an electric pump and a circuit for regulating the pump speed, controlled by a trigger cursor protruding from the gun grip.

The grip is connected to a suction system, a tank or line that supplies the washing liquid, and an electric power supply line.

The terminal or tip in the form of an irrigation and suction rod is generally composed of two concentric pipes, one of which is connected to the pump and provides the washing liquid, while the other is connected to the suction system through the grip.

The end of the irrigation terminal or tip is diverging cone-shaped, so as to limit the area of irrigation.

The pump housed in the grip provides a continuous flow or a pulsating intermittent flow.

These devices for washing surgical sites and wounds present some disadvantages and drawbacks.

The washing fluid, physiological solution, is sprayed in a straight line. Therefore, the washing device must be maneuvered in order to direct the jet of washing water over the whole area of intervention.

The mechanism for controlling and regulating the pump speed is rather complex and expensive to manufacture.

The construction of the grip in the form of a gun requires the study and arrangement of the various internal parts: pump motor, pump impeller, speed regulator, blowing and sucking connections.

The continuous regulation of the flow of washing liquid distracts the operator from the area and from the necessity of specific washing.

An electric connection with compatible contacts and voltages must be available. As the electric motors of the pumps have a low voltage, a specific transformer is required. As the plugs and sockets vary from one model of washing device to another, especially if produced by different firms, a transformer with specific socket and contacts must be available.

To overcome all these inconvenient aspects, a new disposable device for washing wounds and surgical sites has been designed and carried out.

The aim of the new washing device is to irrigate and wash the whole operation site without having to manoeuvre the washing device.

Another aim of the new device is to limit the distraction of the operator as regards the quantity and pressure of the washing liquid.

Another aim of the new device is to be independent of external power supply sources, compatible or incompatible.

Another aim of the new device is to reduce the complexity of construction.

### SUMMARY OF THE INVENTION

These and other direct and complementary aims are achieved by the new disposable device for washing wounds and surgical sites, comprising a cylindrical linear grip, a pump, a switch or mechanism for activating it, a pipe or coupling for the suction system, a pipe for the flow of washing liquid, a set of batteries or accumulators, one or more tips or terminals in the form of straight and/or inclined irrigation and suction rods.

The set of batteries or accumulators is coupled to the inflow pipe for the washing liquid and the electric connection cables are coupled to said inflow pipe.

One of the two ends of the grip is provided with or connected to the pipes for suction, washing liquid inflow and electric power supply, while the opposite end of the grip is equipped with a coupling for the irrigating tip or terminal.

The pump is housed inside the grip, while the switch or activating mechanism protruding from the outer surface of said grip connects the electric power cables to said pump.

The switch or activating mechanism of the pump is such as to suck at only two different speeds, and therefore at two different pressures.

The tips or terminals in the form of irrigation and suction rod are generally composed of two concentric pipes having one end suited to connect and join it to said grip and the opposite end shaped so as to irrigate and to hold the irrigation liquid.

The end of the pipe inside the tip is provided with a sprinkling mechanism that rotates so as to project the washing liquid over the whole area to be washed, while the end of the external suction pipe is shaped in the form of a diverging cone, so as to contain and collect the washing liquid over the whole area washed.

Various tips and terminals are may be provided, straight or with different inclinations.

The new device is provided in a disposable sterile pack which contains various inclined and/or straight tips or ends, as well as the grip and the pack of batteries.

The washing liquid suction pipe in the grip is connected to the suction system present in the operating room.

The washing liquid inflow pipe in the grip is connected to a tank or inlet for the washing liquid.

A suitable tip or terminal is applied on the grip.

When the switch or activating mechanism is operated, the pump that draws the washing liquid and conveys it under pressure to the tip or terminal is started. The pressure of the washing fluid, that is, the pump rotation speed, depends on the specific position (off-slow-fast) of the switch.

At the end of use the whole new device, that is, the grip, batteries, pipes, terminals, can be disposed of without having to be resterilised.

### DETAILED DESCRIPTION OF PREFERRED EMBODIMENT

The characteristics of the new disposable device for washing wounds and surgical sites will be explained in greater detail in the following description of one among many possible implementations of the invention, with reference to the drawings enclosed, wherein:

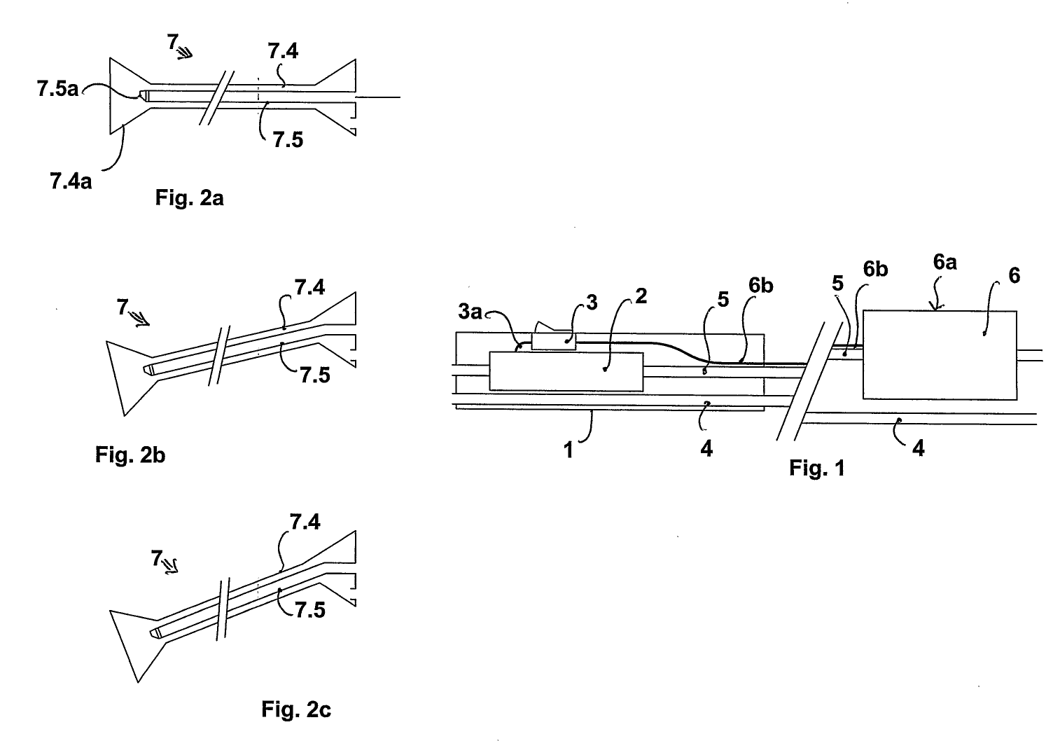

FIG. 1 shows a longitudinal section of the new device, comprising a grip (1), a pump (2), a switch or activating mechanism (3), a pipe (4) or coupling for the suction system, a pipe (5) for the inflow of the washing liquid, a set of batteries or accumulators (6), one or more tips or terminals (7) in the form of an irrigation and suction rod.

The grip (1) comprises a generically cylindrical linear casing, suited to be comfortably gripped and suited to hold the pump (2) and the switch or activating mechanism (3).

In particular, said switch or activating mechanism (3) protrudes or at any rate is accessible from the outside of the grip (1), so that it can be conveniently operated with a finger of the hand holding said grip (1).

At one end of the grip (1) there are the suction pipe (4) and the inflow pipe (5) for the washing liquid. At the opposite end of the grip there is a coupling or mechanism for joining the various irrigating terminals or tips (7) to the grip (1) itself.

The suction pipe (4) passes through the grip (1) and finishes at the end where the various irrigating terminals or tips (7) are joined to the grip (1) itself.

The inflow pipe (5) is connected to the pump (2) and, in sequence, to the end coupling of the grip (1) for connection with the terminals or tips (7). The set of batteries or accumulators (6) is applied on the inflow pipe (5). In particular, said set of batteries or accumulators (6) is included or incorporated in a sealed coating (6a), preferably combined with said inflow pipe (5) in the production phase.

Likewise, an electric cable (6b) is coupled to/incorporated in said inflow pipe (5) and connects the set of batteries or accumulators (6) to the switch or activating mechanism (3).

A further electric cable (3a) connects said switch or activating mechanism (3) to the pump (2).

This switch or activating mechanism (3) has three positions: a first position in which no electric power is supplied to the pump (2), a second position in which the electric power supplied to the pump (2) is such as to make it turn at low speed, a third position in which the electric power supplied to the pump (2) is such as to make it turn at full speed.

Each irrigating terminal or tip (7) mainly comprises two concentric pipes (7.5, 7.4), the internal one (7.5) of which carries the washing liquid, while the external one (7.4) forms, together with the internal pipe (7.5), a suction cavity for the washing liquid.

Substantially, the end of the terminal or tip (7) that connects the grip (1) is such as to put the internal pipe (7.5) in communication with the pump (2) and the cavity between the two pipes (7.5, 7.4) in contact with the suction pipe (4).

The end of the irrigation terminal or tip (7), opposite the grip (1), has a particular shape.

More specifically, the irrigating end of the internal pipe (7.5) is equipped with a rotating sprinkling mechanism (7.5a), in such a way as to project the washing liquid over the whole area to be washed, while the end of the external pipe (7.4) has the shape of a diverging cone (7.4a), so as to hold and collect the washing water only on the area washed.

FIGS. 2a, 2b, 2c illustrate some embodiments of tips or terminals (7) that are straight or with different inclinations.

The new device for washing wounds and surgical sites presents notable advantages.

The linear grip (1) is simpler and less expensive to make. Moreover, the linear grip (1) ensures a better arrangement of its internal parts (switch (3), pump (2)).

The linear grip (1) allows the washing flow to be directed more effectively onto the wound or onto the operation site.

The rotating mechanism (7.5.1) of the irrigating end of the internal pipe (7.5) allows the washing liquid to be projected over the whole area to be washed, with no need to tilt and move the new device.

The only two rotating speeds of the pump (2), that is, the only two pressures of the washing liquid, allow the operation site or the wound to be adequately washed with no need to continuously adjust the pressure on the switch or on the activating mechanism.

Thanks to the presence of the set of batteries or accumulators included in the device, it isn't necessary to use a specific transformer with dedicated contacts and the new device is immediately operative.

Therefore, with reference to the above description and to the enclosed drawings, the following claims are put forth.

1. A device for washing wounds and surgical sites, comprising:
a grip (1) including a pump (2) and a mechanism (3) for activating the pump (2),
an elongated member extending from and coupled to the grip, the elongated member comprising a suction tube (4), and an inflow tube (5), the inflow tube (5) being configured for carrying a washing liquid,
wherein a tip of the inflow tube (5) is rotatable, and
wherein a tip of the suction tube (4) surrounds the rotatable tip of the inflow tube (5).

2. The device for washing wounds and surgical sites according to claim 1, wherein the device is structured to be coupled to interchangeable elongated members, and wherein the interchangeable elongated members are structured to extend from the grip at different angles.

3. The device for washing wounds and surgical sites according to claim 1, further comprising a power source housed in the grip (1), the power source being electrically connected to the mechanism (3) and to the pump (2).

4. (canceled)

5. The device for washing wounds and surgical sites according to claims 1, wherein the mechanism is a switch having at least a first position in which no electric power is supplied to the pump (2), a second position causing the pump to turn at full speed, thereby providing the washing liquid at maximum flow rate, and a third position causing the pump to turn at a speed lower than the full speed, thereby providing the washing liquid at less than the maximum flow rate.

6. (canceled)

7. The device for washing wounds and surgical sites according to claim 1, wherein the tip of the inflow tube is a nozzle.

8. The device for washing wounds and surgical sites according to claim 1, wherein the tip of the suction tube has a wider diameter than the remainder of the suction tube, and wherein the tip of the inflow tube is positioned within the tip of the suction tube.

9. The device for washing wounds and surgical sites according to claim 8, wherein the tip has a frustoconical shape with a smaller base coupled to the suction tube and a larger base extending outwards of the suction tube, and wherein the tip of the inflow tube is positioned within the frustoconical shape.

10. The device for washing wounds and surgical sites according to claim 3, wherein the power source is a battery or a capacitor.

11. The device of claim 10, wherein the power source comprises a plurality of batteries or capacitors.

12. The device for washing wounds and surgical sites according to claim 3, wherein the power source is sealed from the surrounding environment.

13. The device for washing wounds and surgical sites according to claim 12, wherein the power source is sealed with a sealing coating.
